(19) United States
(12) Patent Application Publication
(10) Pub. No.: US 2005/0196456 A1
(43) Pub. Date: Sep. 8, 2005

(54) Controlled release particles

(75) Inventors: Mark T. Anderson, Woodbury, MN (US); Tadesse G. Nigatu, Maplewood, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/029,192

(22) Filed: Jan. 4, 2005

Related U.S. Application Data
(62) Division of application No. 09/920,689, filed on Aug. 2, 2001, now abandoned.
Application No. 09/920,689 is a continuation-in-part of application No. 09/838,854, filed on Apr. 20, 2001, now abandoned.

(51) Int. Cl. B29B 9/00; A61K 9/14

(52) U.S. Cl. 424/489; 264/5

(57) ABSTRACT

A particle that includes an inorganic matrix that includes channels and a composition disposed in the channels, the composition including organic structure-directing agent and active agent, e.g., pheromone, and the particle being capable of controllably releasing the active agent.

### CROSS REFERENCE TO RELATED APPLICATIONS

This application is a divisional of U.S. application No. 09/920,689, filed Aug. 2, 2001; which was a continuation-in-part of U.S. application No. 09/838,854, filed Apr. 20, 2001, now abandoned.

### BACKGROUND OF THE INVENTION

The invention relates to controllably releasing active agent.

It is often desirable to achieve controlled release of an agent. However, the nature of the agent and the environment into which the agent is to be released can make controlled release difficult to achieve. Volatile compounds, for example, present unique problems with respect to controlled release due to their fugitive character. One attempt to achieve controlled release of a volatile compound involves impregnating an absorbent particle with the volatile compound, whereupon the compound is released from the absorbent by vaporizing over a period of time. Such volatile compound-adsorbed particles have also been coated with molten thermoplastic polymers in a further attempt to maintain the compound in the particle until its desired release.

The absorbent particles that are used in these encapsulation techniques are often molecular sieves such as zeolites and surfactant templated particles. Molecular sieves are porous structures and can be found in nature or prepared synthetically. Surfactant templated particles are porous structures that are prepared by condensing an inorganic matrix around a pore forming material or “template”. After the particle is formed, the inorganic matrix is subjected to high temperature calcination or solvent extraction to remove the surfactant. The inorganic matrix that remains has a porous structure, which serves as the absorbent site for the volatile compound.

### SUMMARY

In a first aspect, the invention features a particle that includes an inorganic matrix that includes channels and a composition disposed in the channels, where the composition includes organic structure-directing agent and active agent and the particle is capable of controllably releasing the active agent. In one embodiment, the particle further includes a template that includes the composition. In other embodiments, the inorganic matrix has been formed in the presence of the composition. In some embodiments, the particle has an x-ray diffraction peak less than 6 degrees two theta using copper K_{α} radiation.

In some embodiments, the active agent is hydrophobic. In other embodiments, the active agent is hydrophilic. In another embodiment, the active agent includes pheromone. In other embodiments the active agent is selected from the group consisting of trans-8, trans-10-dedecadien-1-ol, Z-11-tetradecenyl acetate, E-11-tetradecenyl acetate, Z-8-dodecenyl acetate, E-8 dodecenyl acetate, Z-8-dodecenol, Z,Z-3,13-octadecyldienyl acetate, E,Z-3,13-octadecyldienyl acetate and Z-9-dodecenyl acetate and mixtures thereof.

In another embodiment, the active agent includes a curing agent. In some embodiments, the active agent is selected from the group consisting of pharmaceutical agents, therapeutic agents, antimicrobial agents, agricultural agents, hygiene agents, preservatives, disinfectants, and combinations thereof. In one embodiment, the active agent is selected from the group consisting of chlorhexidine digluconate, silver ion, glycerol monolaurate and combinations thereof.

In some embodiments, the active agent is dissolved in the organic structure-directing agent. In other embodiments the active agent is associated with the organic structure-directing agent.

In various embodiments, the particle has an average particle size of no greater than about 20 um, no greater than about 15 um, no greater than about 1000 nm or no greater than about 100 nm.

In another embodiment, the organic structure-directing agent includes surfactant. In some embodiments, the organic structure-directing agent includes latex particles.

In some embodiments, the channels of the particle have an average a cross-sectional dimension no greater than about 50 nm. In other embodiments, the channels of the particle have an average cross-sectional dimension no greater than 30 nm. In some embodiments, the channels are in a substantially parallel relationship. In other embodiments, the channels form an interconnected network.

In one embodiment, the inorganic matrix includes an aggregate of metal oxide particles. In other embodiments, the inorganic matrix includes metal oxide selected from the group consisting of alumina, titania, zirconia and combinations thereof. In some embodiments, the inorganic matrix includes silica.

In a second aspect, the invention features a first composition including a plurality of particles that include an inorganic matrix that includes channels and a second composition disposed in the channels, the second composition including organic structure-directing agent and active agent and the particles being capable of controllably releasing the active agent. In some embodiments, the inorganic matrix was formed in the presence of the second composition.

In other embodiments, the composition further includes a vehicle. In some embodiments, the vehicle is selected from the group consisting of water, alcohols, ketones, aldehydes, nitrites, esters, carboxylates, polyols, hydrocarbons, fluorocarbons and combinations thereof. In another embodiment, the vehicle includes polymerizable monomers. In another embodiment, the vehicle includes a polymer selected from the group consisting of thermoplastic polymer, thermoset polymer, elastomer and combinations thereof. In one embodiment, the vehicle includes epoxy resin and the active agent includes a curing agent. In other embodiments, the vehicle includes an adhesive composition.

In another embodiment, the organic structure-directing agent includes surfactant. In some embodiments, the organic structure-directing agent includes latex particles. In other embodiments, the active agent includes pheromone. In another embodiment, the active agent is selected from the group consisting of trans-8, trans-10-dedecadien-1-ol, Z-11-tetradecenyl acetate, E-11-tetradecenyl acetate, Z-8-dodecenyl acetate, E-8 dodecenyl acetate, Z-8-dodecenol, Z,Z-3,13-octadecyldienyl acetate, E,Z-3,13-octadecyldienyl acetate and Z-9-dodecenyl acetate and mixtures thereof. In other embodiments, the active agent is selected from the group consisting of pharmaceutical agents, therapeutic agents, antimicrobial agents, agricultural agents, hygiene agents, preservatives, disinfectants and combinations thereof. In another embodiment, the active agent is selected from the group consisting of chlorhexidine digluconate, silver ion, glycerol monolaurate and combinations thereof.

In one embodiment, the inorganic matrix includes silica.

In a third aspect the invention features a powder that includes an above-described composition. In some aspects, the invention features a film that includes an above-described composition. In still other aspects, the invention features an article that includes an above-described composition. In one embodiment, the article is of a form selected from the group consisting of a tablet, a pellet and a brick.

In a fourth aspect, the invention features an article that includes a container and a plurality of particles that include an inorganic matrix that includes channels and a composition disposed in the channels, the composition including an organic structure-directing agent and active agent and the particles being capable of controllably releasing the active agent. In one embodiment, the article further includes an aerosol propellant. In another embodiment, the article further includes a vehicle. In some embodiments, the particles are disposed in the vehicle. In other embodiments the vehicle is selected from the group consisting of water, alcohols, ketones, aldehydes, nitriles, esters, carboxylates, polyols, hydrocarbons, fluorocarbons and combinations thereof.

In one embodiment, the active agent is selected from the group consisting of pharmaceutical agents, therapeutic agents, antimicrobial agents, agricultural agents, hygiene agents, preservatives, disinfectants, and combinations thereof. In another embodiment, the active agent includes pheromone.

In a fifth aspect, the invention features a method that includes contacting a target with a first composition that includes a plurality of particles, the particles including an inorganic matrix that includes channels, and a second composition disposed in the channels, the second composition including an organic structure-directing agent and active agent and the particles being capable of controllably releasing the active agent. In one embodiment, the target includes soil. In other embodiments, the target includes a plant. In some embodiments, the target includes a tree.

In one embodiment, the method further includes spraying the first composition. In some embodiments, the first composition further includes a vehicle. In other embodiments, the vehicle is selected from the group consisting of water, alcohols, ketones, aldehydes, nitrites, esters, carboxylates, polyols, hydrocarbons, fluorocarbons and combinations thereof. In some embodiments, the vehicle includes a polymer selected from the group consisting of thermoplastic polymer, thermoset polymer, elastomer and combinations thereof.

In another embodiment, the method further includes exposing the particles to radiation to release the active agent. In some embodiments, the radiation is selected from the group consisting of thermal radiation, ultraviolet radiation and electron beam radiation.

In other embodiments, the active agent includes a curing agent.

In a sixth aspect the invention features a method of making particles capable of controllably releasing an effective amount of an active agent, the method includes forming an inorganic matrix in the presence of a composition that includes an organic, structure-directing agent and active agent. In one embodiment, forming an inorganic matrix includes condensing an inorganic component. In some embodiments, the inorganic component is selected from the group consisting of metal alkoxides, metal carboxylates, metal salts and combinations thereof. In other embodiments, the inorganic component includes alkoxysilane. In another embodiment, the inorganic matrix includes silica.

In one embodiment, the forming an inorganic matrix that includes condensing an inorganic component in the presence of a template includes the composition. In some embodiments, the forming an inorganic matrix includes irreversibly agglomerating colloidal metal oxide particles. In other embodiments, the method further includes drying the agglomerated metal oxide particles. In one embodiment, the organic structure-directing agent includes surfactant. In another embodiment, the organic structure-directing agent includes latex particles. In some embodiments, the method further includes forming the inorganic matrix in the presence of a template that includes the composition.

In a seventh aspect, the invention features a particle that includes an inorganic matrix that includes channels and a composition disposed in the channels, the composition including surfactant and active agent selected from the group consisting of pharmaceutical agents, therapeutic agents, antimicrobial agents, agricultural agents, curing agents, and combinations thereof, the particle being capable of controllably releasing the active agent. In one embodiment, the active agent includes pheromone.

In an eighth aspect, the invention features a composition that includes a plurality of above-described particles.

In a ninth aspect, the invention features an article that includes a container and a plurality of above-described particles disposed in the container. In one embodiment, the article further includes an aerosol propellant.

In a tenth aspect, the invention features a method that includes contacting a target with a first composition that includes a plurality of above-described particles. In various embodiments, the target includes soil, a plant or an apple tree.

In some embodiments, the method further includes spraying the first composition. In other embodiments, the first composition further includes a vehicle. In some embodiments, the vehicle is selected from the group consisting of water, alcohols, ketones, aldehydes, nitrites, esters, carboxylates, polyols, hydrocarbons, fluorocarbons and combinations thereof.

In an eleventh aspect, the invention features a method of making particles capable of controllably releasing an active agent, the method including drying a composition that includes an inorganic component, organic structure-directing agent and active agent. In some embodiments, the method further includes spraying the composition on a substrate prior to drying the composition. In other embodiments, the composition has a pH of from 4 to 9.

In a twelfth aspect, the invention features a method of treating a target that includes a) contacting the target with a composition that includes an inorganic component, organic structure-directing agent and an active agent, and b) drying the composition to form particles capable of controllably releasing the active agent. In one embodiment, the composition has a pH of from 4 to 9. In another embodiment, the method further includes spraying the composition.

In a thirteenth aspect, the invention features a method of making particles capable of controllably releasing an active agent, the method including forming an inorganic matrix in the presence of a composition having a pH of from 4 to 9, the composition including an inorganic component, organic structure-directing agent and the active agent.

The term “structure-directing agent” means an agent that, when in the presence of a condensable inorganic component, is capable of directing the structure that forms as the inorganic component condenses.

The term “active agent” refers to agents that are capable of interacting with a biological system, a chemical system or a combination thereof when released from a particle.

The particles of the present invention can provide controlled release of an active agent under predetermined conditions. The particle can also be formulated to control the rate at which the active agent is released from the composite. The particles can protect an unstable active agent and enhance the shelf life of some active agents.

The particles are particularly well suited for encapsulating volatile active agents and controllably releasing volatile active agents.

In some formulations, the particles can be placed in a liquid vehicle without releasing the active agent contained therein, which enables the particles to be co-formulated with other compounds that require aqueous vehicles and simultaneously applied with those same compounds.

The controlled release particle forming reaction mixture can be formulated to be applied on a substrate, e.g., a target. The controlled release particles form on the substrate as the mixture dries, which facilitates use of the mixture and application of the active agent.

The preparation of controlled release particles from a mixture that includes metal oxide particles and is essentially free of a strong acid or a strong base for catalyzing the formation of the inorganic matrix of the particles can eliminate the need to subsequently remove (e.g., wash away) the strong acid or the strong base from the mixture, which simplifies the particle manufacturing process.

Other features of the invention will be apparent from the following description of the preferred embodiments thereof, and from the claims.

### DETAILED DESCRIPTION

The controlled release particles can be prepared according to various inorganic condensation methods. Inorganic condensation methods generally include condensing an inorganic component in the presence of an organic structure-directing agent and an active agent.

One example of a useful inorganic condensation method includes condensing molecular precursors in the presence of a surfactant template organic structure-directing agent. The method includes preparing a mixture that includes water, surfactant, active agent, a condensable inorganic component, and optionally a condensation catalyst. For purposes of illustration, the method is described with respect to an organic structure-directing agent that is the form of surfactant micelles, however, it is to be understood that the method is suitable for use with other organic structure-directing agents including, e.g., lyotropic surfactant liquid crystals and latex particles. A sufficient amount of surfactant is added to water to form surfactant micelles. The active agent associates with, dissolves in, disperses in or a combination thereof, the surfactant micelles. An aqueous dispersion of condensable inorganic component is then added to the surfactant mixture, which causes the surfactant micelles to agglomerate and form a surfactant template. The condensable inorganic component then surrounds the surfactant template and undergoes a condensation reaction to form the inorganic matrix. The surfactant template around which the inorganic matrix forms, patterns the channels of the inorganic matrix.

Other useful methods of templating particles are described in, e.g., U.S. Pat. No. 5,264,203 and WO 99/37705, and incorporated herein.

Another method of preparing controlled release particles includes adding a condensable inorganic component in the form of colloidal metal oxide particles to a mixture that includes organic structure-directing agent, active agent, and water. As the composition dries, the colloidal metal oxide particles condense, i.e., irreversibly agglomerate, around the organic structure-directing agent to form a solid inorganic matrix that includes channels patterned by the organic structure-directing agent.

The controlled release particles include an inorganic matrix having channels, and a composition that includes organic structure-directing agent and active agent disposed in the channels. The inorganic matrix of the controlled release particle is an inorganic structure and can include a variety of inorganic materials including, e.g., metal oxides (e.g., alumina, silica, titania, zirconia and silicates including, e.g., alumina silicate), metal sulfides, metal (e.g., platinum) and combinations thereof. The inorganic matrix is preferably uncalcined, i.e., the inorganic matrix has not been heated to a temperature sufficient to degrade or decompose organic components present when the inorganic matrix was formed, to substantially dehydroxylate the inorganic matrix, or a combination thereof.

The inorganic matrix is formed from the condensation of condensable inorganic components. Useful condensable, inorganic components include, e.g., molecular precursors, metal salts and inorganic colloidal particles. Useful condensable molecular precursors include, e.g., alkoxides and carboxylates of metal including, e.g., silica, zirconium, titanium, rare earth ions, germanium, tin, tungsten and aluminum. Examples of useful silica precursors include alkoxysilanes including, e.g., tetramethoxysilane, tetraalkoxysilanes including, e.g., tetraethoxysilane, tetrapropoxysilane, tetrabutoxysilane, iso-propoxysilane, sec-butoxysilane, and tert-butoxysilane and combinations thereof, tetrachlorosilane and combinations thereof.

Examples of useful metal salts include metal halides including, e.g., metal chlorides (e.g., iron chloride and aluminum chloride), and metal nitrates including, e.g., iron nitrate and aluminum nitrate.

Suitable colloidal particles include, e.g., metal oxides (e.g., alumina, silica, titania and zirconia), metal sulfides, silicates (e.g., alumina silicate) and combinations thereof. Colloidal silica provides a particularly useful inorganic matrix. Preferred colloidal metal oxide particles have an average particle size from about 2 nm to about 100 nm.

The composition disposed in the channels of the inorganic matrix includes an organic structure-directing agent and active agent. Useful organic structure-directing agents include supramolecular assemblies including, e.g., surfactant micelles and latex particles. The organic structure-directing agent is present during formation of the controlled release particles in an amount sufficient to provide a pattern around which the inorganic matrix condenses during formation of the controlled release particle. A number of organic structure-directing agents can agglomerate to form the pattern around which the inorganic matrix forms. The pattern can be ordered, e.g., a template, or disordered. Preferably the organic structure-directing agent forms an ordered pattern, e.g., a surfactant template or latex template. Preferably the organic structure-directing agent forms an ordered template having properties sufficient to produce a particle having an X-ray diffraction peak of less than 6 degrees two theta using copper K_{α} radiation, which corresponds to a d-spacing of about 15 Angstrom (Å).

The organic structure-directing agent patterns the channels of the inorganic matrix to provide channels that extend through the inorganic matrix in a variety of configurations including, e.g., periodic arrays and random configurations. In one configuration, the channels form a network in which at least some of the channels are interconnected. In other configurations, the channels exist in a parallel arrangement and can include a number of layers of parallel channels. Preferably the majority of the channels of the inorganic matrix extend continuously from one surface of the particle to another such that the entrance to and the exit from a channel is located at the surface of the particle. The channels facilitate diffusion of the active agent out of the particle. The channels of the inorganic matrix preferably have an average cross-sectional dimension, e.g., diameter, from about 1.5 nm to about 2000 nm, preferably from about 1.5 nm to about 50 nm, more preferably from 1.5 nm to about 30 nm. The particles may also include closed cell pores, which may include various components including, e.g., gas (e.g., air), organic structure-directing agent, solvent and combinations thereof.

The organic structure-directing agent is selected to enable the incorporation of a predetermined active agent into the controlled release particle and preferably assists in controlling the release of the active agent from the controlled release particle. Preferably the organic structure-directing agent is selected such that a predetermined active agent dissolves in the organic structure-directing agent. The organic structure-directing agent is also preferably selected to achieve channels having a predetermined cross-sectional diameter as described in more detail below.

The organic structure-directing agent can also be selected to initiate release of the active agent by a factor external to the particle including, e.g., heat, radiation, moisture, solvent, agitation, change in pressure, pH, ionic strength, and combinations thereof. The external factor may also be used to alter the rate at which the active agent is released from the particle.

Suitable organic, structure-directing agents include latex particles and surfactants. Useful latex organic structure-directing agents include, e.g., polystyrene, polymethyl methacrylate, poly(stearyl methacrylate-hexanediol diacrylate) and combinations thereof.

Useful classes of surfactant structure-directing agents include, e.g., cationic, anionic, nonionic and zwitterionic surfactants, fluorosurfactants and combinations thereof.

Suitable cationic surfactants include alkylammonium salts having the formula CₙH₂ₙ₊₁N(CH₃) ₃X, where X is OH, Cl, Br, HSO₄ or a combination of OH and Cl, and where n is an integer from 8 to 22, and the formula CₙH₂ₙ₊₁N(C₂H₅)₃X, where n is an integer from 12 to 18; gemini surfactants, for example those having the formula [Cl₆H₃₃N(CH₃)₂CₘH₂ₘ₊₁]X, wherein m is an integer from 2 to 12 and X is as defined above; and cetylethylpiperidinium salts, for example C₁₆H₃₃N(C₂H₅)(C₅H₁₀)X, wherein X is as defined above. Suitable alkylammonium salts include alkyltrimethylammonium chlorides including, e.g., cetyltrimethylammonium chloride, and alkyltrimethylammonium bromides including, e.g., cetyltrimethylammonium bromide and decyltrimethylammonium bromide. An example of a useful quaternary ammonium salts is ammonium chloride.

Useful anionic surfactants include, e.g., alkyl sulfates having the formula CₙH₂ₙ₊₁OSO₃⁻M⁺, where n is 12 to 18; alkylsulfonates including C₁₆H₃₃SO₃⁻M⁺ and C₁₂H₂₅C₆H₄SO₃⁻M⁺; alkyl phosphates including, e.g., C₁₂H₂₅OPO₃⁻M⁺, and C₁₄H₂₉OPO₃⁻; and alkylcarboxylates including, e.g., C₁₇H₃₅CO₂⁻ and C₁₄H₂₅COOH, where M⁺ is alkali metal ammonium or alkyl ammonium preferably M⁺ is sodium, potassium or ammonium.

Other useful anionic surfactants include, e.g., alkali metal and (alkyl)ammonium salts of alkyl sulfates and sulfonates (e.g., sodium dodecyl sulfate and potassium dodecanesulfonate), sulfates of polyethoxylated derivatives of straight or branched chain aliphatic alcohols and carboxylic acids, alkylbenzene and alkynaphthalene sulfonates and sulfates (e.g., sodium laurylbenzene sulfonate), alkylcarboxylates (e.g., dodecylcarboxylates), ethoxylated and polyethoxylated alkyl and aralkyl alcohol carboxylates, and alkyl phosphates (e.g., ethoxylated dodecyl phosphate).

Useful nonionic surfactants include, e.g., alkylamines including alkylamines having the formula CₙH₂ₙ₊₁NH₂, poly(oxyethylene oxides), poly(octaethylene glycol monodecyl ether) (C₁₂EO₈), poly(octaethylene glyconyl nonhexadecyl ether) (C₁₆EO₈), and poly(alkylene oxide) triblock copolymers such as poly(ethylene oxide)-poly(propylene oxide)-poly(ethylene oxide) and poly(propylene oxide)-poly(ethylene oxide)-poly(propylene oxide). Examples of useful commercially available nonionic copolymer surfactants include nonionic copolymer surfactants available under the trade designation PLURONIC including, e.g., P123, F98, 25R4, 10R5, and 17R4 from BASF Corporation (Mount Olive, N.J.).

Another useful class of surfactants includes ethoxylated amines, which are also referred to as ethoxylated fatty amines. Preferred ethoxylated amines have the formula R—N(CH₂CH₂O)ₓH (CH₂CH₂O)_{y}H, wherein x+y=15 and 50, and are commercially available under the trade designation ETHOMEEN from Akzo Nobel (Chicago, Ill.).

Another suitable class of surfactants includes fluorosurfactants including fluorosurfactants available under the FLUORAD trade designation (e.g., FLUORAD FC-431) from Minnesota Mining and Manufacturing Company (St. Paul, Minn.).

The molecular weight of the organic structure-directing agent and the concentration of organic structure-directing agent in the mixture used to form the controlled release particle can be selected to affect the rate at which the active agent is released from the particle. Increasing the average cross-sectional area of the channels (e.g., average diameter of a generally cylindrical channel) of the inorganic matrix can increase the rate at which the active agent is released from the inorganic matrix and decreasing the average cross-sectional area of the channels can decrease the rate at which active agent is released from the particle. The average cross-sectional area of the channels and the uniformity of the cross-sectional area of the channels can be adjusted by altering the molecular weight of the surfactant. The surfactant molecular weight and the concentration of surfactant in the reaction mixture can be selected based upon the desired cross-sectional area of the channels of the inorganic matrix and the desired uniformity of the cross-sectional area of the channels within the inorganic matrix. Surfactants having relatively shorter alkyl chains, for example, template inorganic matrices having channels with relatively smaller cross-sectional areas, and surfactants having relatively larger alkyl chains template inorganic matrices having channels with relatively larger cross-sectional areas.

Examples of surfactants suitable for preparing composites that include channels having a cross-sectional diameter of from about 1.5 nm to about 5 nm include cationic, anionic, nonionic, zwitterionic and fluorocarbon surfactants having a molecular weight less than about 1000 including, e.g., alkylammonium, phosphate, sulfate and carboxylate surfactants. Surfactants suitable for use in preparing particles that include channels having a cross-sectional diameter of from 5 nm to about 30 nm include neutral or fluorocarbon surfactants having a molecular weight of from about 500 to about 15,000, examples of which include triblock copolymers, e.g., poly(ethylene oxide)-poly(propylene oxide)-poly(ethylene oxide) copolymers and poly(propylene oxide)-poly(ethylene oxide)-poly(ethylene oxide) copolymers available under the trade designation PLURONIC from BASF Corp. (Mount Olive, N.J.).

The concentration of organic structure-directing agent that is present in the particle forming reaction mixture can also affect the cross-sectional diameter of the channels of the controlled release particle. Increasing the concentration, for example, can increase the channel cross-sectional diameter and decreasing respectively the concentration can decrease the channel cross-sectional diameter.

The volume ratio of the organic, structure-directing agent to inorganic matrix in a controlled release particle is preferably from about 10% to about 90%, more preferably from about 25% to about 75%, most preferably from about 40% to about 60%.

The active agent provides a beneficial effect when released from the controlled release particle. The beneficial effect can be direct, indirect or a combination thereof. An active agent such as a pharmaceutical composition can be formulated to provide a beneficial effect on a human being. An active agent such as an agricultural agent can be formulated to have a harmful effect, e.g., killing agricultural pests, and a beneficial effect, e.g., increasing crop yield or preventing the spread of disease to human beings.

Suitable active agents for controlled release include, e.g., pharmaceutical agents, therapeutic agents, antimicrobial agents, agricultural agents (e.g., fertilizers, pesticides, herbicides, insecticides, fungicides, germicide, nutrients, growth inhibitors, growth regulators and pheromones), curing agents (e.g., curatives, crosslinking agents, polymerization agents and catalysts), preservatives, disinfectants, hygiene agents (e.g., oral hygiene agents including, e.g., dentifrices, flavorants and whitening agents, and physical hygiene agents including, e.g., antiperspirants, deodorants and fragrances) and combinations thereof.

Examples of suitable agricultural active agents include menthone and pheromones. Examples of useful pheromones include trans-8, trans-10-dedecadien-1-ol (i.e., codlemone), Z-11-tetradecenyl acetate, E-11-tetradecenyl acetate, Z-8-dodecenyl acetate, E-8 dodecenyl acetate, Z-8-dodecenyl acetate, Z,Z-3,13-octadecyldienyl acetate, E,Z-3,13-octadecyldienyl acetate, and Z-9-dodecenyl acetate and mixtures thereof.

Examples of useful antimicrobial agents include chlorhexidine digluconate, silver ion and glycerol monolaurate.

Useful active agents include hydrophobic and hydrophilic active agents. For controlled release particles that include a surfactant organic structure-directing agent, the active agent is preferably hydrophobic and dissolves in the surfactant such that the active agent is located in the hydrophobic region of the surfactant micelle. Relatively more hydrophilic active agents can associate with the polar ends of the surfactant micelle such that the active agent is located on the exterior of the micelle, i.e., between the micelle and a channel wall in a finished controlled release particle. The controlled release particle can also include a number of active agents each associating with the surfactant according to the same or different mechanism.

The active agent is preferably present in the controlled release particle in an effective amount, i.e., an amount sufficient to produce the desired effect when released from the controlled release particle.

The controlled release particle forming mixture can also include a pore size expanding agent to alter the cross-sectional dimension of the channels of the resulting inorganic matrix. Useful pore size expanding agents include low dielectric constant liquids. Examples of useful low dielectric constant liquids include benzene, alkanes (e.g., linear and branched chain hydrocarbons), alkenes, and aromatics (e.g., mesitylene and toluene) and other low dielectric constant molecules. One example of a useful pore size expanding agent is 1,3,5-trimethylbenzene. Other suitable low dielectric constant liquids are also described in U.S. Pat. No. 5,057,296 (Beck) and incorporated herein.

Combining a pore size expanding agent with a surfactant such as cationic, anionic, nonionic and fluorocarbon surfactants having a molecular weight less than about 1000 (e.g., alkylammonium, phosphate, sulfate and carboxylate surfactants), for example, can produce particles having a pore size of from about 1.5 nm to about 10 nm.

The controlled release particle forming reaction mixture can optionally include a catalyst for catalyzing the formation of the inorganic matrix. Suitable catalysts include, e.g., acids and bases. The acids may be organic or inorganic. Preferred acids include mineral acids including, e.g., hydrochloric, sulfuric, hydrobromic and hydrofluoric acids. Useful bases include alkali metal hydroxides including, e.g., sodium hydroxide, ammonium hydroxide and alkylammonium hydroxides (e.g., tetramethylammonium hydroxide).

Controlled release particle forming reaction mixtures that include colloidal metal oxide particles preferably are essentially free of, more preferably free of, a catalyst such as a strong acid or a strong base. By “essentially free of” is meant that the amount of strong acid or strong base present in the mixture does not require a subsequent washing step to remove the strong acid or strong base. Preferably such particle forming reaction mixtures have a pH of from 4 to 9.

The controlled release particle forming reaction mixture can also optionally include a co-solvent. The co-solvent can be added to the composition to provide a variety of functions including, e.g., improving solution homogeneity, modifying the particle size, modifying the particle morphology, modifying the channel pore size, adjusting the dielectric constant of the particle forming composition, and combinations thereof. The co-solvent can also be added to facilitate dissolution of the active agent in the surfactant. Examples of useful co-solvents include alcohols, esters (for example, acetates), ketones, diols, triols, ethers, amides and amines. Preferred co-solvents include methanol, ethanol, isopropanol, ethylene glycol, formamide, N,N-dimethylformamide, tetrahydrofuran and ethyl acetate.

The ratio of water to polar solvent present in the reaction mixture can be varied depending upon the active agent and surfactant of the composition. Preferably the water:polar solvent ratio is from about 100:0 to about 3:97.

The controlled release particle forming mixture can also include other additives including, e.g., antioxidants. Examples of suitable antioxidants include hindered phenols (e.g., 2,6-di-t-butyl-4-methylphenol), tetrabismethylene 3-(3′,5′-di-tert-butyl-4′-hydroxyphenyl)propionate methane (e.g., IRGANOX 1010 (Ciba-Geigy Corp., Ardsley, N.Y.)), thiodiethylene bis-(3,5-di-tert-butyl-4-hydroxy)hydrocinnamate (e.g., IRGANOX 1035 (Ciba-Geigy Corp.)), N-nitrosophenylhydroxylamine aluminum salt (e.g., Q-1301 (Wako Chemicals USA, Inc. (Richmond, Va.)), hydroquinone and its derivatives including, e.g., methylhydroquinone and polymerized trimethyldihydroquinone, octadecyl-3-(3′,5′-di-tert-butyl-4′-hydroxyphenyl)propionate (IRGANOX 1076, Ciba-Geigy Corp.), distearyl thiodipropionate and combinations thereof.

The controlled release particles preferably have an average cross-sectional dimension of no greater than about 20 μm, preferably no greater than about 15 μm, more preferably no greater than 1000 nm, most preferably no greater than about 100 nm. The controlled release particles preferably have at least one low angle Bragg peak at less than 6° two-theta as determined by X-ray diffraction analysis using copper K_{α} radiation, which corresponds to a d-spacing of about 15 Angstrom (Å).

Controlled release particles can be provided in various forms including, e.g., powder, compositions, films, coatings, agglomerates, composites, pellets and monoliths.

The controlled release particles can also be formulated with other components including, e.g., binders and vehicles. A binder can be included in the formulation to facilitate or maintain the controlled release particles in a predetermined form including, e.g., tablet, pellet or brick. Examples of suitable binders include polymers, starches, gums and clays.

A vehicle can be selected to facilitate application of the controlled release particles to a target or handling of the particles. The vehicle can also be part of a system in which release of active agent into the vehicle causes a desired interaction (e.g., reaction) with the vehicle. Examples of such systems include an active agent that is a curing agent (e.g., a crosslinking agent, a polymerization initiator and combinations thereof) and a vehicle that is curable, e.g., polymerizable, crosslinkable and combinations thereof. Upon release, the active agent polymerizes or crosslinks the polymer to form a cured composition. Examples of useful curing agents include polymerization initiators, crosslinking agents and combinations thereof.

Examples of useful curable vehicles include acrylic acid, epoxy resins, urethane resins and combinations thereof. Other useful vehicles include, e.g., water, alcohols, ketones, aldehydes, nitriles, esters, carboxylates, polyols, hydrocarbons, fluorocarbons and combinations thereof, thermoplastic resins, thermoset resins, polymerizable resins, crosslinkable resins, latex compositions and moisture curable compositions.

The controlled release particles are useful in a wide variety of applications including, e.g., agriculture, forestry, medicine, adhesives, coatings, sealants, cosmetics and fragrances. The controlled release particles can be formulated to be useful in treating targets such as soil, plants (e.g., trees), mammals and substrates, and to release active agent over a predetermined period of time. Preferably the active agent is released in an effective amount to achieve a desired result.

The controlled release particles can be applied to a target using methods such as spraying, coating and extrusion. One useful method includes spraying the liquid particle forming reaction mixture on a substrate, e.g., a target or a particle collection surface, and allowing the liquid of the mixture to evaporate, whereupon controlled release particles and aggregates of controlled release particles form on the substrate.

The controlled release particles can be stored in containers and formulated with an aerosol propellant to facilitate spray application.

The invention will now be described further by way of the following examples. All parts, ratios, percents and amounts stated in the Examples are by weight unless otherwise specified.

### EXAMPLES

Test Procedures

Test procedures used in the examples include the following.

Gas Chromatographic Method for Determining Codlemone Content

A sample is prepared by placing approximately 0.3-0.4 g of sample to in a thistle tube and then adding approximately 1.5 ml ethyl acetate to the sample. The sample is then crushed by pressing an inner ground glass rod against the sample in the tube. The crushed sample is then transferred to a 10.0 ml volumetric flask by rinsing multiple times with ethyl acetate. The sample is brought to volume using ethyl acetate. The sample is left to extract on the bench top at room temperature for 20 minutes. The sample is then filtered through a 0.2 μm filter and injected on a codlemone calibrated Hewlett Packard 5890 gas chromatograph (Hewlett Packard Corp., Palo Alto, Calif.) to determine codlemone content.

The sample is injected into a 1 microliter split injection port that is heated to 250° C. and has a split ratio of 20:1. The sample is carried through an open tube 30 m×0.32 mm capillary column having a 5% phenyldimethylsilane coating on a HP5 0.25 μm thick stationary phase (Agilent) with a helium carrier gas flowing at a constant rate of 3.5 ml/min and a head pressure of 14.8 psi. After injection, the oven temperature is ramped from 50° C. to 300° C. at a rate of 20° C./min. The sample is detected using flame ionization detector at 325° C.

Antimicrobial Test Method

Staphylococcus epidermidis expressing luxAB is grown overnight at 37° C. in tryptic soy broth that includes chloramphenicol (10 ug/ml). A small aliquot of the culture is placed in a disposable cuvette and the optical density is measured at 600 nm in a UV spectrophotometer (Beckman Coulter Company, Fullerton, Calif.) to determine concentration. The culture is diluted to obtain a working concentration of approximately 4×10⁶ per ml. A 250 μl sample of diluted bacteria is placed evenly across the surface of a 13 mm nylon filter having a pore size of 0.45 micron (Millipore) and filtered by vacuum. The resulting bacteria-coated filter is placed on top of a culture plate containing 3% tryptic soy agar with the bacteria-coated surface facing outward. The filter/agar plates are allowed to equilibrate for 10 min.

Samples are then cut into 15 mm disks with a hollow punch and hammer. The test samples (5 mg) and control materials (5 mg) are placed over the bacteria-coated filters and a “time zero” reading is recorded immediately with a CCD camera system (Hamamatsu). Before each camera reading, the bacteria are exposed for 3 minutes to decanal (Sigma) vapors. This is accomplished by replacing the original petri dish cover with one that contained a thin film of 1% decanal in ethanol on its inner surface. This provides the bacteria with a source of aldehyde substrate to facilitate the generation of light.

Each CCD camera reading includes collecting both a brightfield image integrated over two minutes with an external light source and a bioluminescent image from bacteria-generated light. These two images are combined into a computer-assisted superimposed image where the magnitude of bioluminescent light is designated with pseudocolor and superimposed onto the brightfield image. The intensity of the light photons from each imaged sample is determined by identifying a “region of interest” with computer software tools and then recording the resulting relative intensity units. The system is quantified using standard curves.

If the relative intensity unit reflects that the bioluminescent bacterial growth was unchanged or unimpeded, a “0” is assigned. If at least 99% of the bioluminescent bacteria are killed, a “+” is assigned.

X-Ray Diffraction Determination

X-ray diffraction data is collected using a Kratky camera (Anton Paar, Graz, Austria) equipped with copper K_{α} rotating radiation anode (Rigaku, Tokyo, Japan) and a linear position sensitive detector (M. Braun, Munich, Germany). The samples are contained within 1.0 mm glass capillary tubes and a transmission geometry is employed. Scans are run from 0 to 10 degrees two theta. The positions of diffraction peaks are determined through JADE software (Materials Data Inc., Livermore, Calif.).

Thermogravimetric Analysis

Approximately 20 mg of the powder (dry) sample is place on a tared platinum pan. The pan is introduced into a Thermogravimetric Analysis Instrument (TA Instruments, New Castle, Del.). The sample is ramped from room temperature to 950° C. at 10° C./min. The resulting weight loss versus temperature data are analyzed with Universal Analysis Software (TA Instruments, New Castle, Del.) to determine total organic content (in grams/gram of sample). The data for washed samples is then compared to the unwashed sample to determine the percentage of organic released upon washing (% organic lost on washing=(g organic in washed sample/gram of washed sample)/(g organic in unwashed sample/gram of unwashed sample)).

### Example 1

The particles of Example 1 were prepared by combining, with mixing, 0.3 g butylatedhydroxytoluene, 20 g 10% by weight codlemone in ethanol, 20 g 10% by weight PLURONIC 123 ethyleneoxide-propyleneoxide-ethyleneoxide surfactant (BASF Corp., Mount Olive, N.J.) in deionized water and 50.7 g NYACOL 2034DI 34% by weight colloidal silica (having a mean particle diameter of approximately 20 nm as reported by the manufacturer) in water (Akzo Nobel Company, Marietta, Ga.). The mixtures were mixed on a roller mill for 30 minutes. The controlled release particle-containing liquid compositions were then dried, whereupon aggregates of controlled release particles formed.

### Example 2

The particles of Example 2 were prepared as described in Example 1 with the exception that the composition also included 58 g CAT 80 42% by weight acid stabilized aqueous colloidal solution of silica and alumina (having a particle size of approximately 80 nm as reported by the manufacturer) (Akzo Nobel) and did not include NYACOL 2034DI colloidal silica. The controlled release particle-containing liquid compositions were then dried, whereupon aggregates of controlled release particles formed.

### Example 3

The particles of Example 3 were prepared as described in Example 1 with the exception that the composition of Example 3 included 0.6 g butylatedhydroxytoluene and 58 g NYACOL 2034DI colloidal silica. The controlled release particle-containing liquid compositions were then dried, whereupon aggregates of controlled release particles formed.

### Example 4

The particles of Example 4 were prepared as described in Example 1 with the exception that the composition of Example 4 included 1.0 g butylatedhydroxytoluene and 56 g NYACOL 2034DI colloidal silica. The controlled release particle-containing liquid compositions were then dried, whereupon aggregates of controlled release particles formed.

### Control 1

Control 1 composition was prepared by combining 20 g 10% codlemone in ethanol and 50 g NYACOL 2034DI colloidal silica.

Sample aliquots were removed from the compositions of Examples 1-4 and Control 1 prior to drying. The samples were aged by placing them in a dish exposed to the atmosphere. After overnight exposure to air, the liquid of the samples had evaporated, leaving a visible film residue. The aged samples were then tested according to the Gas Chromatographic Method for Determining Codlemone Content. Results are shown in Table 1.

TABLE 1

| | % by Weight of Initial Codlemone Concentration | | | | | | | |
|---|---|---|---|---|---|---|---|---|
| Sample | 0* | 1* | 9* | 14* | 21* | 28* | 33* | 42* |
| Control 1 | 100 | 2.0 | — | 0.6 | 0.001 | 0.0008 | — | — |
| Example 1 | 100 | 102 | 93 | 73 | 72 | 24 | 2 | 0.3 |
| Example 2 | 100 | 7.1 | 7.3 | 4.7 | 1.0 | 0.6 | 0.02 | 0.01 |
| Example 3 | 100 | 112 | 105 | 120 | 105 | 72 | 62 | 41 |
| Example 4 | 100 | 88 | 114 | 106 | 93 | 76 | 55 | 37 |

“—” = not measured.
*= Number of elapsed days.

### Example 5

A particle composition was prepared by mixing in a glass jar: 103 g 10% by weight PLURONIC P123 surfactant in deionized water, 15 g concentrated ammonium hydroxide, 50 g 10% weight codlemone in ethanol solution, 15 g ethanol and 41 g NALCO 1042 colloidal silica. The composition was stirred for 20 hours at room temperature, placed in an oven at 40° C. for 24 hours and then filtered and dried. A first portion of the composition was maintained at room temperature and a second portion of the composition was heated to 40° C. The % by weight codlemone remaining in the compositions after 7, 14, 28 and 42 days was measured according to the Gas Chromatographic Method for Determining Codlemone Content. The results in % by weight codlemone are reported in Table 3.

TABLE 3

| Example | Days | Room Temperature | 40° C. |
|---|---|---|---|
| 5 | 0 | 5.5 | 5.5 |
| 5 | 7 | 5.2 | 3.5 |
| 5 | 14 | 4.6 | 2.6 |
| 5 | 28 | 2.9 | 1.3 |
| 5 | 42 | 3.1 | 0.6 |

### Example 6

Example 6 was prepared by combining 20 g epoxy curative 2-ethyl-4-methyl-2-imidazole with 10 g of a 10% by weight aqueous PLURONIC P123 surfactant. The mixture was then combined and mixed with 70 g NYACOL 2034DI colloidal silica. The mixture was then mixed on a roller mill for 3 hours, allowed to settle overnight, and then exposed to air to allow the water to evaporate. The composition formed a white brittle sheet, which was crushed into a powder with a mortar and pestle.

3 g of the powder (about 1.4 g of catalyst) was mixed with 10 g EPON 828 bisphenol A diglicidyl ether epoxy resin (Shell Chemical Co., Houston, Tex.) and placed in a 45° C. oven for 24 hrs. When examined the next day the composition was liquid.

The temperature of the liquid was then raised to 100° C. and held at 100° C. overnight. When examined the next day, the composition had hardened.

### Comparative Example 1

The composition of Comparative Example 1 was prepared by mixing 1 g 2-ethyl-4 methyl 2-imidazoline powder and 10 g EPON 828 epoxy resin and placed in the 45° C. oven for 24 hrs. When examined the next day the composition had hardened to a solid.

### Example 7

A composition having particles with a molar ratio of 1 tetramethoxysilane:0.13 cetyltrimethylammonium bromide:18.1 methanol:96 water:0.10 NH₄OH:0.2 menthone ((C₈H₁₇₀) potato sprout inhibitor) was prepared by mixing 0.2 g cetyltrimethylammonium bromide, 7.40 g deionized, water, 2.45 g methanol, 0.049 g 30% by weight ammonium hydroxide and 0.15 ml menthone in a 20 ml vial. To this mixture was added 0.625 ml tetramethoxysilane with vigorous stirring. Within about 10 seconds a milky white dispersion formed. A stable dispersion persisted for more than 1 year.

A portion of the composition was filtered to remove the particles. The filtered particles were then examined after one week and the smell of menthone was detected.

### Example 8

A composition having particles with a molar ratio of 1 tetramethoxysilane:0.13 cetyltrimethylammonium bromide:22.7 methanol:92.6 water:0.10 NH₄OH:0.13 codlemone were prepared by mixing 2.0 g cetyltrimethylammonium bromide, 70 g deionized water, 39.1 ml methanol, 0.49 g 30% by weight ammonium hydroxide and 1 g E,E-8,10 dodecadien-1-ol (codlemone, a pheromone for the Codling moth) in a 125 ml polypropylene bottle. Then 6.25 ml tetramethoxysilane were added to the mixture. The mixture was magnetically stirred at 900 rpm and within about 15 seconds the mixture formed a milky white dispersion of particles.

### Example 9

A composition having particles with a molar ratio of 1 tetramethoxysilane:0.13 dodecyltrimethylammonium bromide:22.7 methanol:92.6 water:0.10 NH₄OH:0.13 codlemone were prepared according to Example 13 with the exception that the particles were prepared using dodecyltrimethylammonium bromide instead of cetyltrimethylammonium bromide. A stable milky white dispersion of particles formed.

### Example 10A-10C

The particles of Examples 10A, 10B and 10C were prepared by mixing 0.2 g cetyltrimethylammonium bromide, 4.8 g water 3.9 ml methanol, 0.1 g codlemone, 3.5 ml 30% ammonium hydroxide and 0.625 ml tetramethoxysilane. The compositions were stirred at 1400 rpm and gelled after approximately 2 seconds to form a whitish opaque gel.

Several hours after preparation: Example A was suction filtered and washed on filter paper with water, Example B was filtered, washed with water and then washed with 300 ml methanol, and Example C was filtered and washed with 300 ml of 75:25 water:methanol solution.

Control 2

Control 2 was prepared according to Examples 10A-10C with the exception that no codlemone was added to the mixture. After preparation, Control 2 was filtered and washed with 300 ml water.

Examples 10A-10C and Control 2 were tested according to the Thermogravimetric Analysis test method. The results revealed that for Example A nearly all of the codlemone and surfactant remained in the particles, for Example B about 30% of the codlemone plus surfactant had been washed from the particles, for Example C 93% of the codlemone plus surfactant remained in the particles, and for Control 2 all of the surfactant remained in the particles.

### Example 11A-F

Six particle compositions were prepared from solutions having a constant 65:35 w/w water:methanol ratio and a variable codlemone: cetyltrimethylammonium bromide (CTAB) ratio by combining, with mixing, water, methanol, cetyltrimethylammonium bromide, codlemone, and 30% by weight NH₄OH in the amounts set forth in Table 4. The compositions of Examples 11A-11C were clear. The compositions of Examples 11D-11F formed emulsions.

Tetramethoxysilane was then added to the compositions with rapid stirring. The compositions of Examples 11A-11C formed gels in 2 to 3 seconds. The compositions of Examples 11D-11E formed a white suspension. Example 11F formed a white fluid dispersion.

Control 3

A 0.6% by weight codlemone mixture in 65:35 water:methanol solution immediately formed an emulsion.

Examples 11A-F and Control 3 were filtered, dried and then tested using X-ray diffraction. The X-ray diffraction results are set forth in Table 4.

TABLE 4

| | | | | | | 30% by | | |
|---|---|---|---|---|---|---|---|---|
| | | | | | | weight | | d₁₀₀ spacing |
| | Water | methanol | CTAB | codlemone* | | NH₄OH | | from X-ray |
| Example | (g) | (ml) | (g) | (g) | Codlemone:CTAB | (ml) | Tetramethoxysilane | diffraction |
| 11A | 4.8 | 3.9 | 0.2 | — | 0 | 3.5 | — | 35.2 |
| 11B | 4.8 | 3.8 | 0.16 | 0.1 | 0.25 | 3.5 | 0.625 | 36.1 |
| 11C | 4.8 | 3.7 | 0.12 | 0.2 | 0.66 | 3.5 | 0.625 | 38.3 |
| 11D | 4.8 | 3.6 | 0.08 | 0.3 | 1.6 | 3.5 | 0.625 | 37.9 |
| 11E | 4.8 | 3.5 | 0.04 | 0.4 | 4 | 3.5 | 0.625 | 38.6 |
| 11F | 4.8 | 3.4 | — | 0.5 | Infinity | 3.5 | 0.625 | —¹ |

*20% by weight in methanol
— = not present
¹= This sample was not ordered and thus did not have a measurable low angle Bragg peak.

### Example 12

A glass jar was charged with 8 g PLURONIC P123 surfactant dissolved in an acidic solution of 46 g hydrochloric acid and 210 g distilled water, 10 g chlorhexidine digluconate and 17 g tetraethoxysilane. The composition was stirred at 35° C. overnight. The composition formed particles, which were isolated by filtration.

Control 4

A glass jar was charged with an acidic solution of 46 g hydrochloric acid and 210 g distilled water and 10 g chlorhexidine digluconate. The composition was stirred at 35° C. overnight.

Control 5

A glass jar was charged with an acidic solution of 46 g hydrochloric acid and 210 g distilled water, 10 g chlorhexidine digluconate, 130 g stearyl acrylate and 17 g tetraethoxysilane. The composition was stirred at 35° C. overnight.

Control 6

A glass jar was charged with an acidic solution that included 23 g hydrochloric acid and 105 g distilled water and 8 g tetraethoxysilane. The composition was stirred at 35° C. overnight. The composition formed particles, which were isolated by filtration.

The particles of Example 12 were tested according to the Antimicrobial Test Method and the results are reported in Table 5, where “0”=no bacterial kill and “+”=at least 99% bacterial kill.

Example 12 initially demonstrated no antibacterial activity and at three hours exhibited at least 99% bacterial kill. Example 12 and Controls 4 and 5 were then tested at 5 hours and 7 hours at which points there was no additional bacteria growth and the samples continued to exhibit at least 99% bacterial kill.

Control 6, which contained no chlorhexidine digluconate, showed no decrease in bacterial bioluminescence for the length of the experiment (7 hours).

TABLE 5

| Sample | Initial | 3 Hour | 5 Hour | 7 Hour |
|---|---|---|---|---|
| Example 12 | 0 | + | + | + |
| Control 4 | + | + | + | + |
| Control 5 | + | + | + | + |
| Control 6 | 0 | 0 | 0 | 0 |

### Example 13

A particle composition was prepared with codlemone according to Example 10. The composition was filtered and the filtered particles were placed in 3″×5″ tared metal pans.

Over the course of 60 days, Example 13 was periodically examined for the smell of codlemone. Throughout the periodic examinations and at 60 days codlemone was detected in Example 13, which indicated that codlemone was being released for at least 60 days.

Other embodiments are within the claims.

1. A method of making particles capable of controllably releasing an active agent, said method comprising
forming an inorganic matrix in the presence of a composition comprising organic structure-directing agent and active agent.

2. The method of claim 1, wherein forming an inorganic matrix comprises condensing an inorganic component.

3. The method of claim 2, wherein said inorganic component is selected from the group consisting of metal alkoxides, metal carboxylates, metal salts and combinations thereof.

4. The method of claim 2, wherein said inorganic component comprises alkoxysilane.

5. The method of claim 1, wherein said inorganic matrix comprises metal oxide selected from the group consisting of alumina, titania, zirconia, and combinations thereof.

6. The method of claim 1, wherein said inorganic matrix comprises silica.

7. The method of claim 1, wherein said forming an inorganic matrix comprises condensing an inorganic component in the presence of a template comprising said composition.

8. The method of claim 1, wherein said forming an inorganic matrix comprises irreversibly agglomerating colloidal metal oxide particles.

9. The method of claim 8, further comprising drying said agglomerated metal oxide particles.

10. The method of claim 1, wherein said organic structure-directing agent comprises surfactant.

11. The method of claim 1, wherein said organic structure-directing agent comprises latex particles.

12. The method of claim 1, further comprising forming said inorganic matrix in the presence of a template comprising said composition.

13. The method of claim 1, wherein said composition further comprises antioxidant.

14. A method of making particles capable of controllably releasing an active agent, said method comprising drying a composition comprising an inorganic component, organic structure-directing agent and active agent.

15. The method of claim 14, further comprising spraying said composition on a substrate prior to drying said composition.

16. The method of claim 14, wherein said composition has a pH of from 4 to 9.
